(19) United States
(12) Patent Application Publication
(10) Pub. No.: US 2004/0242729 A1
(43) Pub. Date: Dec. 2, 2004

(54) Stabilized particle dispersions containing surface-modified inorganic nanoparticles

(75) Inventors: Jimmie R. Baran JR., Prescott, WI (US); Brian J. Gabrio, Oakdale, MN (US); James S. Stefely, Woodbury, MN (US); Stephen W. Stein, Lino Lakes, MN (US); Thomas E. Wood, Stillwater, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/449,359

(22) Filed: May 30, 2003

(51) Int. Cl. C08K 9/00; A61K 9/00

(52) U.S. Cl. 523/200; 424/400

(57) ABSTRACT

This invention relates to particle-in-liquid dispersions containing surface-modified inorganic nanoparticles.

### BACKGROUND

This invention relates to particle-in-liquid dispersions.

Traditional dispersions are made up of two phases: a dispersed phase and a continuous phase. The most common dispersions consist of only dispersed particles and a liquid continuous phase. If the formed dispersion is not stabilized, the dispersed particles will flocculate or agglomerate and the two phases will separate. Typically, dispersants are used to prevent the two phases from separating. Dispersants stabilize dispersions through steric or electrostatic means after being adsorbed onto the dispersed particles. Increasing the viscosity of the continuous phase may also prevent complete phase separation of dispersions.

### SUMMARY

In one aspect, the invention provides dispersions comprising a dispersed phase and a continuous phase. The dispersed phase comprises particles dispersed in the continuous phase. The continuous phase comprises a liquid continuous phase and surface-modified inorganic nanoparticles.

In another aspect, the invention provides a method of stabilizing a dispersion comprising adding an effective amount of compatible surface-modified inorganic nanoparticles to a dispersion comprising a dispersed phase comprising particles and a continuous phase comprising a liquid.

In another aspect, the invention provides a pharmaceutical dispersion wherein the dispersed phase comprises one or more medicaments.

In another aspect, the invention provides a method for treating a mammal comprising administering a therapeutically effective amount of a medicament dispersion to the mammal orally, by injection, through its nasal passage, by inhalation, topically, or combinations thereof.

In another aspect, the invention provides a dispersion kit comprising a dispersed phase component to be dispersed in a continuous phase and surface modified inorganic nanoparticles.

### DETAILED DESCRIPTION

The dispersions of the invention are stable dispersions that remain dispersed over useful time periods without substantial agitation or which are easily redispersed with minimal energy input. The dispersions comprising insoluble particles and a continuous phase are rendered stable by incorporation of an effective amount of surface-modified inorganic nanoparticles into the continuous phase. An “effective amount” of surface-modified nanoparticles is an amount that minimizes the aggregation of the dispersed particles and forms stable dispersions that remain dispersed over a useful time period without substantial agitation of the dispersion or which are easily redispersed with minimal energy input. Without wishing to be bound by any theory, the nanoparticles are believed to sterically inhibit the aggregation of the dispersed phase and not through particle charge. The surface-modified nanoparticles stabilize the dispersions without the use of conventional dispersants. The dispersions of the invention may contain less than 0.001 percent by weight of surfactant, surface-active agents, detergents, and/or conventional dispersants as those terms are used in the art.

As used herein, “dispersion” means solid particles distributed or suspended within a liquid continuous phase which does not separate over a useful time period, for example, minutes, hours, days, etc. As used herein, “dispersion” means solid particles distributed or suspended within a liquid continuous phase which does not separate over a useful time period, for example, minutes, hours, days, etc.

As used herein, “separate” means that the solid particles in a liquid dispersion gradually settle or cream, forming distinct layers with very different concentrations of the solid particles and continuous liquid phase.

As used herein, “dispersion stability” is a description of the tendency of a dispersion to separate. For a dispersion with good dispersion stability, the particles remain approximately homogeneously distributed within the continuous phase. For a dispersion with poor dispersion stability, the particles do not remain approximately homogeneously distributed within the continuous phase and may separate.

As used herein, an “excipient” refers broadly to any inert additive other than the primary active medicament moiety used to improve some aspect of the aerosol dispersion formulation.

Stabilized dispersions of the invention include surface-modified inorganic nanoparticles. The surface-modified nanoparticles are preferably individual, unassociated (i.e., non-aggregated) nanoparticles dispersed throughout the continuous phase and preferably do not irreversibly associate with each other or with the dispersed particles. The term “associate with” or “associating with” includes, for example, covalent bonding, hydrogen bonding, electrostatic attraction, London forces, and hydrophobic interactions.

The surface-modified nanoparticles are selected such that the composition formed therewith is free from a degree of particle agglomeration or aggregation that would interfere with the desired properties of the composition. The surface-modified nanoparticles are selected to be compatible with the liquid continuous phase.

One method of assessing the compatibility of the surface-modified nanoparticles with the liquid continuous phase includes determining whether the resulting composition separates. For transparent liquid continuous phases, one useful method of assessing the compatibility of the surface-modified nanoparticles with the transparent liquid continuous phase includes the step of combining the surface-modified nanoparticles and the liquid continuous phase and observing whether the surface-modified nanoparticles completely disperse in the liquid continuous phase. Since the nanoparticles have dimensions smaller than the wavelength of visible light, complete dispersion will result in a transparent dispersion.

Since the inorganic component of the surface-modified nanoparticles is chosen to be insoluble in the liquid continuous phase, the surface-modified nanoparticles will disperse, but not dissolve in that phase. The surface modification of the particle will allow it to be compatible with the liquid phase so that it can completely disperse. When the nanoparticles are smaller than the wavelength of visible light, the nanoparticles will appear to form a transparent solution when completely dispersed. As the size of the surface-modified nanoparticles increases, the haziness of the continuous phase generally increases. Desirable surface-modified nanoparticles are selected such that they do not settle out of the continuous phase.

The further step in assessing the compatibility of the continuous phase and the surface-modified nanoparticles includes determining whether, upon subsequent introduction of liquid to be dispersed in the continuous phase, the composition forms a stable dispersion phase in a useful period of time. A useful period of time may be minutes, hours, days, weeks, or years, depending upon the application. For example, when the dispersion of the invention is a pigment, it is desirable for the dispersion to remain stable for months. However if the dispersion of the invention is a pharmaceutical in a formulation, it may only be necessary for the dispersion to remain stable for several minutes, until the pharmaceutical is administered.

Suitable surface groups can also be selected based upon the solubility parameter of the surface group and the continuous phase. Preferably the surface group, or the agent from which the surface group is derived, has a solubility parameter similar to the solubility parameter of the continuous phase. When the continuous phase is hydrophobic, for example, one skilled in the art can select from among various hydrophobic surface groups to achieve a surface-modified particle that is compatible with the hydrophobic continuous phase. Similarly, when the continuous phase is hydrophilic, one skilled in the art can select from hydrophilic surface groups, and, when the continuous phase is a hydrofluorocarbon, one skilled in the art can select from among various compatible surface groups. The nanoparticle can also include at least two different surface groups that combine to provide a nanoparticle having a solubility parameter that is similar to the solubility parameter of the continuous phase. The surface-modified nanoparticles are not amphiphilic.

The surface groups may be selected to provide a statistically averaged, randomly surface-modified particle.

The surface groups are present on the surface of the particle in an amount sufficient to provide surface-modified nanoparticles that are capable of being subsequently dispersed in the continuous phase without aggregation. The surface groups preferably are present in an amount sufficient to form a monolayer, preferably a continuous monolayer, on the surface of the nanoparticle.

Surface modifying groups may be derived from surface modifying agents. Schematically, surface modifying agents can be represented by the formula A-B, where the A group is capable of attaching to the surface of the particle and the B group is a compatibilizing group that may be reactive or non-reactive with a component of the continuous phase. Compatibilizing groups can be selected to render the particle relatively more polar, relatively less polar or relatively non-polar.

Suitable classes of surface-modifying agents include, e.g., silanes, organic acids organic bases and alcohols, and combinations thereof.

Particularly useful surface-modifying agents include silanes. Examples of useful silanes include organosilanes including, e.g., alkylchlorosilanes, alkoxysilanes, e.g., methyltrimethoxysilane, methyltriethoxysilane, ethyltrimethoxysilane, ethyltriethoxysilane, n-propyltrimethoxysilane, n-propyltriethoxysilane, i-propyltrimethoxysilane, i-propyltriethoxysilane, butyltrimethoxysilane, butyltriethoxysilane, hexyltrimethoxysilane, octyltrimethoxysilane, 3-mercaptopropyltrimethoxysilane, n-octyltriethoxysilane, phenyltriethoxysilane, polytriethoxysilane, vinyltrimethoxysilane, vinyldimethylethoxysilane, vinylmethyldiacetoxysilane, vinylmethyldiethoxysilane, vinyltriacetoxysilane, vinyltriethoxysilane, vinyltriisopropoxysilane, vinyltrimethoxysilane, vinyltriphenoxysilane, vinyltri(t-butoxy)silane, vinyltris(isobutoxy)silane, vinyltris(isopropenoxy)silane, and vinyltris(2-methoxyethoxy)silane; trialkoxyarylsilanes; isooctyltrimethoxy-silane; N-(3-triethoxysilylpropyl) methoxyethoxyethoxy ethyl carbamate; N-(3-triethoxysilylpropyl) methoxyethoxyethoxyethyl carbamate; silane functional (meth)acrylates including, e.g., 3-(methacryloyloxy)propyltrimethoxysilane, 3-acryloyloxypropyltrimethoxysilane, 3-(methacryloyloxy)propyltriethoxysilane, 3-(methacryloyloxy)propylmethyldimethoxysilane, 3-(acryloyloxypropyl)methyldimethoxysilane, 3-(methacryloyloxy)propyldimethylethoxysilane, 3-(methacryloyloxy)methyltriethoxysilane, 3-(methacryloyloxy)methyltrimethoxysilane, 3-(methacryloyloxy)propyldimethylethoxysilane, 3-(methacryloyloxy)propenyltrimethoxysilane, and 3-(methacryloyloxy)propyltrimethoxysilane; polydialkylsiloxanes including, e.g., polydimethylsiloxane, arylsilanes including, e.g., substituted and unsubstituted arylsilanes, alkylsilanes including, e.g., substituted and unsubstituted alkyl silanes including, e.g., methoxy and hydroxy substituted alkyl silanes, and combinations thereof.

Methods of surface-modifying silica using silane functional (meth)acrylates are described, e.g., in U.S. Pat. Nos. 4,491,508; 4,455,205; 4,478,876; 4,486,504; and 5,258,225, and incorporated herein.

Useful organic acid surface-modifying agents include, e.g., oxyacids of carbon (e.g., carboxylic acid), sulfur and phosphorus, and combinations thereof.

Representative examples of polar surface-modifying agents having carboxylic acid functionality include CH₃O(CH₂CH₂O)₂CH₂COOH (hereafter MEEAA) and 2-(2-methoxyethoxy)acetic acid having the chemical structure CH₃OCH₂CH₂OCH₂COOH (hereafter MEAA) and mono(polyethylene glycol) succinate in either acid or salt forms.

Representative examples of non-polar surface-modifying agents having carboxylic acid functionality include octanoic acid, dodecanoic acid and oleic acid.

Examples of suitable phosphorus containing acids include phosphonic acids including, e.g., octylphosphonic acid, laurylphosphonic acid, decylphosphonic acid, dodecylphosphonic acid, octadecylphosphonic acid, and monopolyethylene glycol phosphonate in either acid or salt forms.

Useful organic base surface-modifying agents include, e.g., alkylamines including, e.g., octylamine, decylamine, dodecylamine, octadecylamine, and monopolyethylene glycol amines.

Examples of other useful non-silane surface modifying agents include acrylic acid, methacrylic acid, beta-carboxyethyl acrylate, mono-2-(methacryloyloxyethyl) succinate, and combinations thereof. A useful surface modifying agent that imparts both polar character and reactivity to the nanoparticles is mono(methacryloyloxypolyethyleneglycol) succinate.

Examples of suitable surface-modifying alcohols include, e.g., aliphatic alcohols including, e.g., octadecyl, dodecyl, lauryl and furfuryl alcohol, alicyclic alcohols including, e.g., cyclohexanol, and aromatic alcohols including, e.g., phenol and benzyl alcohol, and combinations thereof.

When the continuous phase includes aromatic ring containing epoxy resins, useful surface-modifying groups can include an aromatic ring. Examples of surface-modifying groups particularly suitable for epoxy resin compositions are disclosed in U.S. Pat. No. 5,648,407, and incorporated herein.

A variety of methods are available for modifying the surface of nanoparticles including, e.g., adding a surface modifying agent to nanoparticles (e.g., in the form of a powder or a colloidal dispersion) and allowing the surface modifying agent to react with the nanoparticles. One skilled in the art will recognize that multiple synthetic sequences to bring the nanoparticle together with the compatibilizing group are possible and are envisioned within the scope, e.g., the reactive group/linker may be reacted with the nanoparticle followed by reaction with the compatibilizing group. Alternatively, the reactive group/linker may be reacted with the compatibilizing group followed by reaction with the nanoparticle. Other useful surface modification processes are described in, e.g., U.S. Pat. Nos. 2,801,185 and 4,522,958, and incorporated herein.

The nanoparticles are inorganic. Examples of suitable inorganic nanoparticles include silica and metal oxide nanoparticles including zirconia, titania, calcium phosphate, e.g., hydroxy-apatite, ceria, alumina, iron oxide, vanadia, antimony oxide, tin oxide, alumina/silica, and combinations thereof, and include combined materials such as a mixture of materials or layers of materials surrounding a central inorganic core. The nanoparticles have an average particle diameter less than about 100 nm, in other embodiments, no greater than about 50 nm; from about 3 nm to about 50 nm; from about 3 nm to about 20 nm; and from about 5 nm to about 10 nm. The ranges include any size or range in between 3 nm and less than 100 nm. If the nanoparticles are aggregated, the maximum cross-sectional dimension of the aggregated particle is within any of these preferable ranges.

Useful surface-modified zirconia nanoparticles include a combination of oleic acid and acrylic acid adsorbed onto the surface of the particle.

Useful surface-modified silica nanoparticles include silica nanoparticles surface-modified with silane surface modifying agents including, e.g., acryloyloxypropyl trimethoxysilane, 3-methacryloyloxypropyltrimethoxysilane, 3-mercaptopropyltrimethoxysilane, n-octyltrimethoxysilane, isooctyltrimethoxysilane, and combinations thereof. Silica nanoparticles can be treated with a number of surface modifying agents including, e.g., alcohol, organosilane including, e.g., alkyltrichlorosilanes, trialkoxyarylsilanes, trialkoxy(alkyl)silanes, and combinations thereof and organotitanates and mixtures thereof.

The nanoparticles may be in the form of a colloidal dispersion. Examples of useful commercially available unmodified silica starting materials include nano-sized colloidal silicas available under the product designations NALCO 1040, 1050, 1060, 2326, 2327, and 2329 colloidal silica from Nalco Chemical Co., Naperville, Ill.

Useful metal oxide colloidal dispersions include colloidal zirconium oxide, suitable examples of which are described in U.S. Pat. No. 5,037,579, and incorporated by reference herein, and colloidal titanium oxide, useful examples of which are described in PCT Publication No. WO 00/06495, entitled, “Nanosize Metal Oxide Particles for Producing Transparent Metal Oxide Colloids and Ceramers,” (Arney et al.), filed Jul. 30, 1998, and incorporated by reference herein.

The stabilized dispersions of the invention comprise a liquid continuous phase. The continuous phase may be made up of one or more miscible or soluble non-reactive constituents so long as the dispersed particles may be dispersed in the liquid continuous phase resulting from the utilized ratio of the constituents of the continuous phase.

Example liquid continuous phases include water, organic liquids including, e.g., acids, alcohols, ketones, aldehydes, amines, amides, esters, glycols, ethers, hydrocarbons, halocarbons, monomers, oligomers, lubricating oils, vegetables oils (including mono- di, and tri-glycerides), silicone oils, moisturizing oils (for example, mineral and jojoba oils), fuel oils, fuels (including kerosene, gasoline, diesel fuel), oligomers of ethylene glycol, alkyl and aryl nitro compounds, partially or fully fluorinated compounds, and polymers, and combinations thereof. In some embodiments, the liquid continuous dispersions may be at least 95, 90, 85, 80, 75, 70, 65, 60, 55, 50, 45, 40, 35, 30, 25, 20, 15, 10, 5 weight percent water and may be any range between 100 and 0 weight percent water. In some embodiments, the liquid continuous dispersions may be at least 95, 90, 85, 80, 75, 70, 65, 60, 55, 50, 45, 40, 35, 30, 25, 20, 15, 10, 5 weight percent organic and may be any range between 100 and 0 weight percent organic.

The continuous phase may have additional components dissolved in it that do not affect the stability of the dispersion (aid or hinder the dispersion of the dispersed insoluble particles), for example, excipients that affect the biologic suitability, salts or organic materials or other beneficial properties of the dispersion.

The dispersed phase may be any particle of interest that have minimal solubility in the liquid continuous phase. Desirably, the particles have a maximum diameter of less than about 100 micrometers. The dispersed particles may be inorganic, organic, or a combination thereof. Examples of dispersed particles include medicaments, carbon black, titanium dioxide, exfolients, cosmetics, pigments, and abrasives.

Specific medicaments include antiallergics, analgesics, bronchodilators, antihistamines, therapeutic proteins and peptides, antitussives, anginal preparations, antibiotics, anti-inflammatory preparations, diuretics, hormones, or sulfonamides, such as, for example, a vasoconstrictive amine, an enzyme, an alkaloid or a steroid, and combinations of these specific examples of medicaments which may be employed are: isoproterenol, phenylephrine, phenylpropanolamine, glucagon, adrenochrome, trypsin, epinephrine, ephedrine, narcotine, codeine, atropine, heparin, morphine, dihydromorphinone, dihydromorphine, ergotamine, scopolamine, methapyrilene, cyanocobalamin, terbutaline, rimiterol, salbutamol, isoprenaline, fenoterol, oxitropium bromide, reproterol, budesonide, flunisolide, ciclesonide, formoterol, fluticasone propionate, salmeterol, procaterol, ipratropium, triamcinolone acetonide, tipredane, mometasone furoate, colchicine, pirbuterol, beclomethasone, beclomethasone dipropionate, orciprenaline, fentanyl, diamorphine, and dilitiazem. Others are antibiotics, such as neomycin, cephalosporins, streptomycin, penicillin, procaine penicillin, tetracycline, chlorotetracycline and hydroxytetracycline; adrenocorticotropic hormone and adrenocortical hormones, such as cortisone, hydrocortisone, hydrocortisone acetate and prednisolone; antiallergy compounds such as cromolyn sodium, nedocromil protein and peptide molecules such as insulin, pentamidine, calcitonin, amiloride, interferon, LHRH analogues, IDNAase, heparin, etc. If applicable, the medicaments exemplified above may be used as either the free base or as one or more salts known to the art. Vaccines may also benefit from this approach.

The medicaments exemplified above may be used as either the free base or as one or more salts known to the art. The choice of free base or salt will be influenced by the physical stability of the medicament in the formulation. For example, it has been shown that the free base of salbutamol exhibits a greater dispersion stability than salbutamol sulphate in the formulations of the invention.

The following salts of the medicaments mentioned above may be used: acetate, benzenesulphonate, benzoate, bicarbonate, bitartrate, bromide, calcium edetate, camsylate, carbonate, chloride, citrate, dihydrochloride, edetate, edisylate, estolate, esylate, fumarate, fluceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylbromide, methylnitrate, methylsulphate, mucate, napsylate, nitrate, pamoate (embonate), pantothenate, phosphatediphosphate, polygalacturonate, salicylate, stearate, subacetate, succinate, sulphate, tannate, tartrate, and triethiodide.

Cationic salts may also be used. Suitable cationic salts include the alkali metals, e.g., sodium and potassium, and ammonium salts and salts of amines known in the art to be pharmaceutically acceptable, e.g., glycine, ethylene diamine, choline, diethanolamine, triethanolamine, octadecylamine, diethylamine, triethylamine, 1-amino-2-propanol-amino-2-(hydroxymethyl)propane-1,3-diol and 1-(3,4-dihydroxyphenyl)-2 isopropylaminoethanol.

For pharmaceutical purposes, the particle size of the medicament powder should desirably be no greater than 100 micrometers diameter. In another embodiment, the particle size should be less than 25 micrometers in diameter. Desirably, the particle size of the finely-divided solid powder should for physiological reasons be less than about 25 micrometers and preferably less than about 10 micrometers in diameter.

Medicinal dispersions according the present invention contain a medicament dispersed in the dispersion in a therapeutically effective amount. “Therapeutically effective amount” means an amount sufficient to induce a therapeutic effect, such as bronchodilation or antiviral activity. The amount will vary according to factors know to those skilled in the art, such as pharmacological activity of the particular medicament, the condition being treated, the frequency of administration, the treatment site, and the presence of any other therapeutic agents or excipients being co-administered. The concentration of medicament depends upon the desired dosage but is generally in the range of 0.01 to 15, 0.01 to 10; 0.01 to 5; 0.01 to 4; 0.01 to 3; or 0.01 to 2 percent by weight and may be present in any amount or range between 0.001 and 15 percent by weight.

The medicinal dispersions of the invention may be delivered to the patient (mammal) by administration means including orally, injection (for example, IV, IP, IM, subQ), topical, through its nasal passage, by inhalation, and combinations thereof. Medicament delivery devices known to those skilled in the art may be used to administer the pharmaceutical dispersion. Such devices include for example, pump sprays, nebulizers, syringes, and the like.

Dispersion kits of the invention comprise surface modified inorganic nanoparticles and a dispersed phase component. The purpose of such a kit is to allow an end user of the dispersion to form the dispersion by adding a continuous phase, at a time the end user desires. The kit could contain pre-determined amounts of dispersed phase component and surface modified nanoparticles to be mixed with a suitable amount of a continuous phase. The dispersed phase component and the nanoparticles may be supplied as powders/particles, or pre-dispersed in a liquid medium. The nanoparticles and the dispersed phase component may be supplied in the kit mixed together or separately. The kit may also further comprise directions for use by the end user, for example, amounts, ratios, useful continuous phases, mixing steps, and the like, to form a dispersion of the invention.

The dispersions and dispersion kits of the invention may also contain surface modified organic molecules, unmodified organic molecules, and/or organic polymeric nanoparticles in combination with surface-modified inorganic nanoparticles. Surface-modified organic molecules, unmodified organic molecules, and organic polymeric microspheres are described in U.S. application Ser. No. ______, (Attorney Docket No. 58588US002), filed on May 30, 2003, incorporated herein by reference for the description of the surface-modified organic molecules, unmodified organic molecules and organic polymeric nanoparticles.

The invention will now be described further by way of the following examples.

### EXAMPLES

Preparation of Iso-octyl Surface Modified Silica Nanoparticles (IO-nano SiO₂)

Iso-octylsilane surface modified silica nanoparticles (IO-nano SiO₂) were prepared as described in U.S. Patent Publication No. 2002/0128336, incorporated by reference herein for said preparation.

### Examples 1-6

Dispersions of insoluble particles of either carbon black, aluminum oxide, and cerium oxide were prepared by combining in individual screw cap vials 0.1 gram (g) of each insoluble solid and 1.9 g of 2% 10-nano SiO₂ in toluene (Example 2 in U.S. Patent Publication No. 2002/0128336). Additional samples were prepared with 0.25 g of solid and 1.9 g of 2% 10-nano SiO₂ in toluene. The vials were then capped and were shaken vigorously by hand for 15 seconds. The vials were then allowed to stand for 5 minutes, after which the suspension of the solids was noted. Suspensions were judged to be stable if the solids remained suspended for 5 minutes. The data are given in Table 1.

### Comparative Examples A-C

Comparative samples A-C were formulated exactly the same way as Examples 1, 3, and 5, respectively (carbon black, aluminum oxide, and cerium oxide) with the difference being that the surface modified silica particles were omitted from the formulation. All of the comparative suspensions were not stable; the insoluble solids settled out of the liquid phase in less than 5 minutes.

TABLE 1

| | | Wt. of | |
|---|---|---|---|
| Example | Suspended Solid | Suspended Solid | Stable Suspension |
| 1 | Carbon black | 0.1 g | Yes |
| 2 | Carbon black | 0.25 g | Yes |
| 3 | Aluminum oxide | 0.1 g | Yes |
| 4 | Aluminum oxide | 0.25 g | Yes |
| 5 | Cerium oxide | 0.1 g | Yes |
| 6 | Cerium oxide | 0.25 g | Yes |

### Examples 7-15

Composition I

250 g. Nalco 2326 (colloidal silica dispersion, available from Nalco Chemicals, Naperville, Ill.), 46.3 g Silquest A1230 (available from Crompton Chemicals, Middlebury, Conn.), and 203.5 g of ultrapure water were mixed and heated at 80° C. for 18 hours.

Composition II

6.7 g of Composition I was added to ajar and combined with 93.3 g ultrapure water.

Composition III

15 mL of Composition II was added to a 50 mL volumetric flask and diluted to volume with ultrapure water.

Composition IV

5 mL of Composition II was added to a 50 mL volumetric flask and diluted to volume with ultrapure water.

The formulations for Examples 7-15 are shown below in Table 2. Samples were prepared by adding a known amount of beclomethasone dipropionate (BDP) into a glass vial and adding 10 mL of one of the nanoparticle compositions described below. The vials were capped, shaken for about 30 seconds, left undisturbed for 20 minutes, and the dispersion stability characteristics were observed and recorded.

TABLE 2

| | Composition | BDP |
|---|---|---|
| Sample | (10 mL) | Amount (g) |
| Example 7 | II | 0.1028 |
| Example 8 | II | 0.0504 |
| Example 9 | II | 0.0101 |
| Example 10 | III | 0.1013 |
| Example 11 | III | 0.0508 |
| Example 12 | III | 0.0104 |
| Example 13 | IV | 0.1015 |
| Example 14 | IV | 0.0499 |
| Example 15 | IV | 0.0104 |
| Comparative | Ultrapure | 0.1010 |
| Example A | H₂O | |
| Comparative | Ultrapure | 0.0507 |
| Example B | H₂O | |
| Comparative | Ultrapure | 0.0098 |
| Example C | H₂O | |

Visual Comparison Results of Comparative Examples A-C and Examples 7-15

The visual comparison results for Examples 7, 10, and 13 and Comparative Example A were as follows: Comparative Example A had very little medicament dispersed within the liquid continuous phase. The majority of the medicament remained on the surface of the liquid continuous phase or on the walls of the vial above the liquid surface. Examples 7, 10, and 13 appeared to have much more medicament dispersed in the liquid continuous phase than Comparative Example A and less medicament on the surface of the liquid or walls of the vial than Comparative Example A. Comparing Examples 7, 10, and 13, higher concentrations of surface-modified nanoparticles provided dispersions appearing to have higher levels of dispersed medicament.

The above observations were also true for Examples 8, 11, and 14 and Comparative Example B and Examples 9, 12, and 15, and Comparative Example C.

All patents, patent applications, and publications cited herein are each incorporated by reference, as if individually incorporated. Foreseeable modifications and alterations of this invention will be apparent to those skilled in the art without departing from the scope and spirit of this invention. This invention should not be restricted to the embodiments that are set forth in this application for illustrative purposes.

What is claimed is:

1. A dispersion comprising:
a continuous phase comprising a liquid continuous phase and surface modified inorganic nanoparticles; and
a dispersed phase comprising particles dispersed in the liquid continuous phase.

2. The dispersion of claim 1 wherein the liquid continuous phase comprises an organic liquid.

3. The dispersion of claim 1 wherein the liquid continuous phase comprises water.

4. The dispersion of claim 1 wherein the liquid continuous phase comprises at least 50 percent by weight water.

5. The dispersion of claim 1 wherein said individual nanoparticles have a particle diameter no greater than about 50 nanometers.

6. The dispersion of claim 1 wherein said individual nanoparticles have a particle diameter in the range of from about 3 nanometers to about 50 nanometers.

7. The dispersion of claim 1 wherein said individual nanoparticles have a particle diameter of no greater than about 20 nanometers.

8. The dispersion of claim 1 wherein said individual nanoparticles have a particle diameter in the range of from about 3 nanometers to about 20 nanometers.

9. The dispersion of claim 1 wherein said individual nanoparticles have a particle diameter in the range of from about 3 nanometers to about 10 nanometers.

10. The dispersion of claim 1 wherein said nanoparticles are selected from the group consisting of silica, titania, alumina, zirconia, vanadia, calcium phosphate, ceria, iron oxide, antimony oxide, tin oxide, aluminum/silica, and combinations thereof.

11. The dispersion of claim 1 wherein said nanoparticles comprise surface groups selected from the group consisting of hydrophobic groups, hydrophilic groups, and combinations thereof.

12. The dispersion of claim 1 wherein the nanoparticles comprise hydrophilic surface groups selected from the group consisting of polyethylene glycols.

13. The dispersion of claim 1 wherein said nanoparticles comprise surface groups derived from an agent selected from the group consisting of silane, organic acid, organic base, and combinations thereof.

14. The dispersion of claim 1 wherein said nanoparticles comprise organosilyl surface groups derived from an agent selected from the group consisting of alkylsilane, arylsilane, alkoxysilane, and combinations thereof.

15. The dispersion of claim 1 wherein said nanoparticles comprise surface groups derived from an agent selected from the group consisting of carboxylic acids, sulfonic acids, phosphonic acids, and combinations thereof.

16. The dispersion of claim 1 wherein the liquid continuous phase is selected from the group consisting of water, organic acids, alcohols, ketones, aldehydes, amines, amides, esters, glycols, ethers, hydrocarbons, halocarbons, monomers, oligomers, lubricating oils, vegetable oils, silicone oils, mineral and jojoba oils, fuel oils, kerosene, gasoline, diesel fuel, oligomers of ethylene glycol, alkyl and aryl nitro compounds, partially or fully fluorinated compounds, and combinations thereof.

17. The dispersion of claim 1 wherein the dispersed phase is one or more medicaments.

18. The dispersion of claim 17 wherein the liquid continuous phase comprises water, ethanol, propylene glycol, glycerol, lactate esters, or combinations thereof.

19. The dispersion of claim 17 wherein the liquid continuous phase further comprises dissolved inorganic or organic salts, polymers, excipients, or combinations thereof.

20. The dispersion of claim 17 wherein the liquid continuous phase is at least 50% by weight water.

21. The dispersion of claim 17 wherein the medicament is selected from the group consisting of steroids, antibiotics, bronchodilators, or analgesics.

22. The dispersion of claim 1 which comprises less than 0.001 percent by weight of surfactant.

23. The dispersion of claim 1 which comprises less than 0.001 percent by weight of surfactant, surface-active agents, detergents, and conventional dispersants.

24. The dispersion of claim 1 further comprising organic nanoparticles.

25. The dispersion of claim 17 which comprises less than 0.001 percent by weight of surfactant, surface-active agents, detergents, and conventional dispersants.

26. A method of stabilizing a dispersion comprising adding an effective amount of compatible surface-modified inorganic nanoparticles to a dispersion comprising a dispersed solid phase and a liquid continuous phase.

27. A method for treating a mammal comprising administering a therapeutically effective amount of the medicament dispersion according to claim 17 to the mammal by administration means selected from the group consisting of orally, injection, topically, through its nasal passage, by inhalation, and combinations thereof.

28. The method of claim 26 wherein the administration of the effective amount of the medicament dispersion is by inhalation using a nebulizer.

29. The method of claim 26 wherein the administration of the effective amount of the medicament dispersion is by nasal passage or topically using a pump spray.

30. The method of claim 26 wherein the administration of the effective amount of the medicament dispersion is by injection.

31. A dispersion kit comprising a dispersed phase component to be dispersed in a continuous phase and surface modified inorganic nanoparticles.
